(19) United States
(12) Patent Application Publication
(10) Pub. No.: US 2006/0173301 A1
(43) Pub. Date: Aug. 3, 2006

(54) Novel method of analysis and measurement of the distribution or reserve of the coronary blood flow

(76) Inventor: Yves Darlas, Blois (FR)

Correspondence Address: HEDMAN & COSTIGAN P.C., 1185 AVENUE OF THE AMERICAS, NEW YORK, NY, 10036, US

(21) Appl. No.: 11/206,397

(22) Filed: Aug. 18, 2005

(30) Foreign Application Priority Data
Jan. 6, 2005 (FR) 0500111

(51) Int. Cl. A61B 6/00

(52) U.S. Cl. 600/436

(57) ABSTRACT

The present invention relates to the field of the essentials of life and, more particularly, to the field of biological analysis. More particularly it relates to a method for the analysis, detection and/or prognosis of disturbances of blood flow in the different regions of the organism, in patients capable of demonstrating alterations in blood flow in different organs of the body, in particular in coronary flow, in which the blood flow is measured before and after a physical, medicinal and/or neurosensory stimulation test, which modifies the blood flow in the region considered after administration of a tracer the fixation of which is measured, so that its elimination rate is determined with respect to a control value using calculation software. Use for testing cerebral, cardiac or tumorous vascularization disturbances.

The present invention relates to the field of the essentials of life and, more particularly, to the field of measuring certain biological parameters.

More particularly it relates to a method for the analysis, detection and/or prognosis of disturbances of circulatory flow in the different regions of the organism, in patients capable of demonstrating alterations in blood flow in different organs of the body, and in particular in coronary flow.

Specifically it relates to a method of measuring and analyzing blood flow during a normal examination, with or without the addition of an agent which increases or modifies blood flow, which consists of measuring the blood flow before and after a physical, medicinal and/or neurosensory stimulation test, which modifies the blood flow in the defined region after administration of a tracer the fixation of which is measured, so that its elimination rate is determined with respect to a control value using calculation software.

The method of measuring and analyzing the results according to the invention has the great advantage of being able to determine disturbances in the blood flow in different organs of the body, such as for example the brain or the heart, as well as in tumorous cells. It is therefore possible to detect abnormalities well before pathological symptoms are noticed and therefore to be able to take the therapeutic or surgical measures which are required, as a preventive or prophylactic means.

More precisely, as regards coronary flow, the time during which the tracer is fixed on the cardiac apparatus and its effect on coronary flow are determined. This method involves no additional operation on the patient. The method only comprises, but not limitatively, the collection of digital data, their processing and their analysis so as to establish a prognostic or therapeutic evaluation of the functioning of the organs considered, and in particular of the heart.

In this way, the risk of accidents is avoided, in particular at the cardiac level, linked to exertion tests and the consequences of which, in certain cases, prove to be dramatic. To this end, an administration, and in particular an injection, of a pharmacodynamic agent is carried out which modifies the blood flow, either by increasing it, or by reducing it and the variations in the blood flow are measured.

A pharmacological agent which increases the blood flow is, for example, dipyridamole, adenosine or dobutamine.

A pharmacological agent which reduces the blood flow is, for example, atropine or its derivatives

Thus, the injection of a pharmacological agent which increases the blood flow has the effect of increasing the flow in the arteries, and this increase, in particular as regards heart beat, manifests itself as during an exertion test, but this effect is rapid and transient, such that it can be easily controlled.

The simultaneous or consecutive administration of a tracer allows the development and concentration of such a product to be monitored during the test. The tracer must essentially have a determination ability by fixing on the tissues and by allowing the evaluation of its elimination or circulation rate. The fixation of the tracer can occur according to a so-called cellular or extracellular “capillary compartment” model.

It is known, in fact, that the vascular system, and above all the cardiac apparatus, constitutes a closed system, the tissue distribution kinetics of which for a given time, such as the tissue distribution time, can be assimilated into a closed compartmental kinetic model, arranged in the circulatory flow.

The tracer used in the method according to the invention is constituted by a gamma ray emitter, and in particular a positon emitter.

The tracer can also be a fluorescent product or a product capable of exhibiting fluorescence, such as for example cosine, fluorescein, erythrosine.

The tracer can also be a contrast product, such as for example an iodinated product, such as for example Telebrix, Telebrix meflumine or lopamiron.

The contrast product used as a tracer is advantageously a contrast product which is visible by medical imaging (MRI), such as for example a derivative of gadolinium.

The contrast product used as a tracer can also be a product which is detectable by ultrasonography or by ultrasonic Doppler effect, such as for example Ecovist or Levovist.

After injection, the fixation of the tracer in the region concerned is determined, depending on the case, using an X-ray imaging device, such as for example a gamma camera, an X-ray scanner, a positon emission tomography camera, or any MRI imaging device.

According to the invention, the tracer is preferably a radiopharmaceutical or a positon emitter or a thallium isotope tracer. A radiopharmaceutical tracer can also be advantageously used chosen from the radioactive isotopes of technetium, and in particular metastable technetium Tc⁹⁹, the radioactive isotopes of indium and the radioactive isotopes of iodine.

Preferably, a radioactive tracer labelled with metastable Technetium Tc⁹⁹, such as Tetrufosmine or the product called MIBI (Dupont Pharma) is used as tracer. Measurement of the fixation and measurement of the circulatory flow of the tracer allows an elimination rate to be calculated with respect to a control value constituted by the parameters of the patient, determined under the same conditions, without administration of a pharmacological agent increasing or reducing the circulatory flow. This measurement is carried out using a calculation software, such as for example that filed by the Applicant at the Program Protection Agency in Paris (France) under the number IDDN FR 001-150011-000-SP2004/00 and called “CORYFLOW”.

The method according to the invention has a particularly valuable use for the determination or the detection of early cardiac disturbances, well before the appearance of any pathological phenomena, such as for example in diabetic subjects who present a significant risk factor.

The method according to the invention also has a significant use for studying vascularization phenomena, in particular for studying cerebral blood circulation, either after a cerebral vascular accident, or earlier when a hypertensive or atherosclerotic patient can present symptoms of a reduction in the blood perfusion of the brain.

Moreover, the method according to the invention has a significant use for studying the vascularization of tumorous tissues. It is known, in fact, that the tumorous tissues are frequently congested as a result of the anarchic development of a neovascularization. Any measure which leads to a reduction of the vascularization in such an area can lead to an absence of perfusion in this region, and therefore, a reduction in the size of the tumorous tissue.

The method according to the invention will be defined more precisely on the basis of the operating method detailed below.

The same analysis method can also be of use for the determination or the quantification of other biological parameters. The attached flow diagram provides an explanation of the use of the calculation software.

The same analysis method can also be applied to the determination of the perfusion of other tissues, by the use of appropriate tracers.

### BRIEF DESCRIPTION OF THE DRAWINGS

FIG. 1 is a graph of the raw data of the choice of the boundaries.

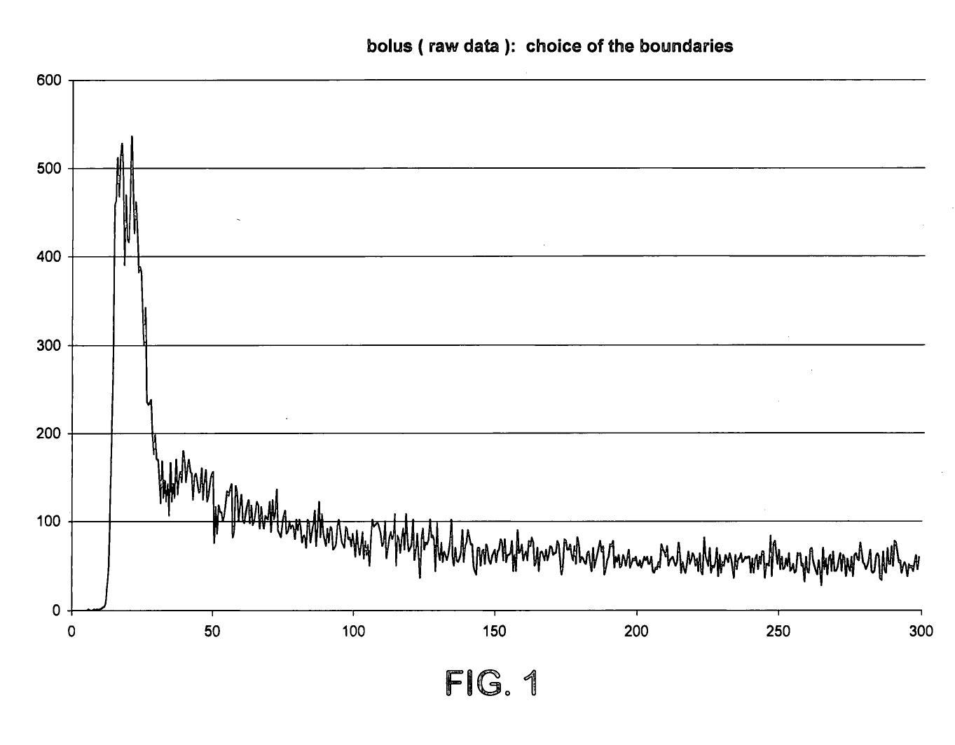

FIG. 2 is a graph of the raw data bolus and processed bolus.

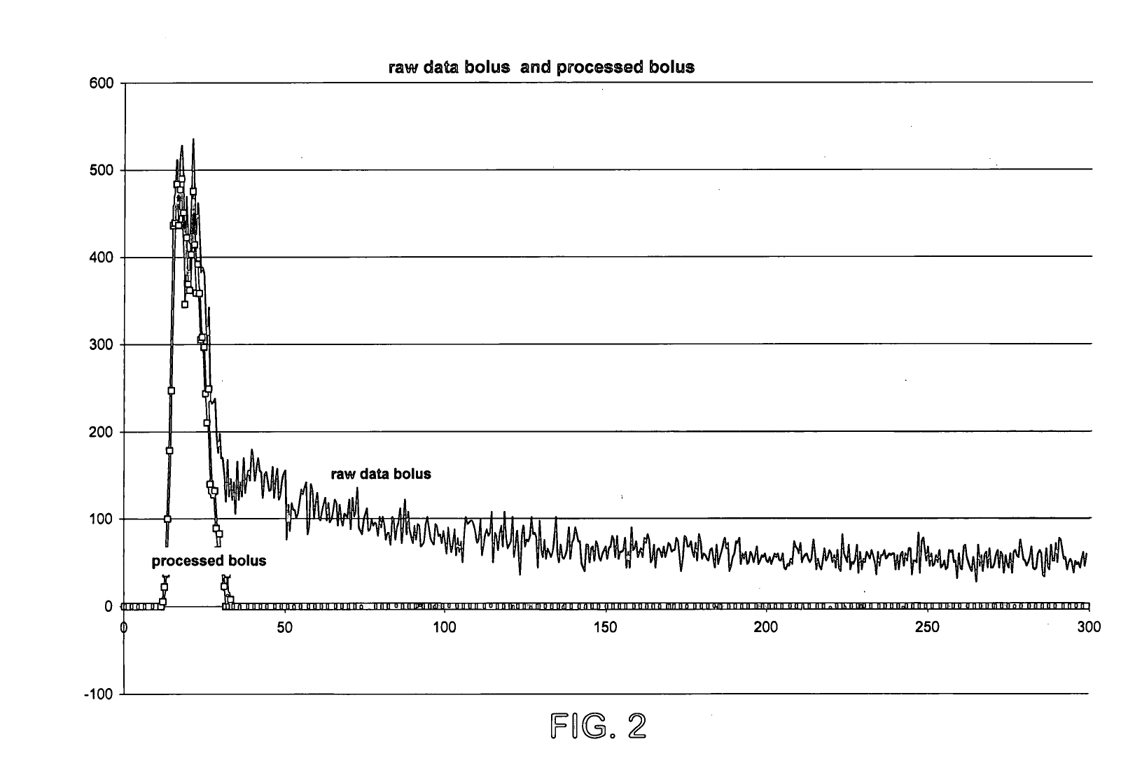

FIG. 3 is a table of the digital values obtained for a patient.

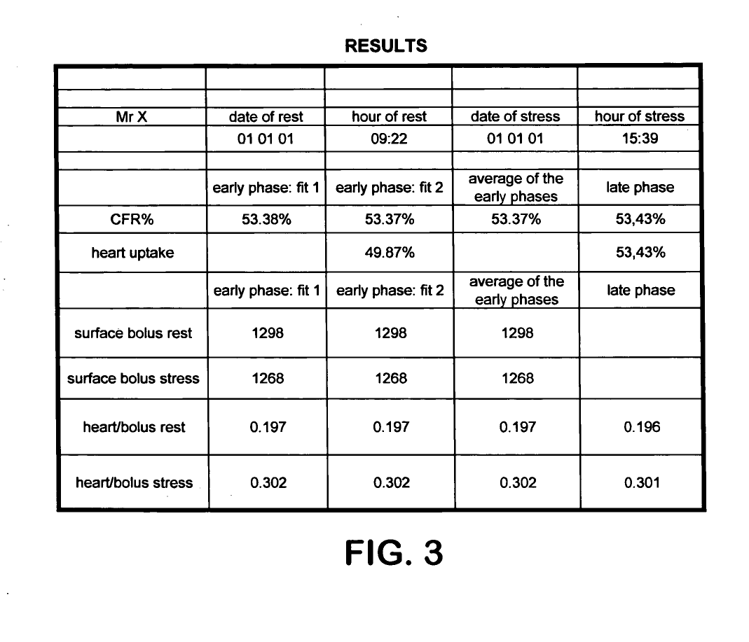

### OPERATING METHOD

Insert the CD-ROM into the computer,
Leave to spin for a short while
Then open “My Computer”
Click on MID
Then go to the name of a patient (for example called Mr. X)
With the mouse, right-click on “send to . . . ” then “My documents”.
Then remove the CD-ROM
Then go to “My documents”.
Go to “Mr. X”
Use “copy” then “paste” a good ten times (or twenty times, or more) in order to obtain as many copies as there are patients to treat
IMPORTANT NOTE: Only work on of the copies, as the file “Mr. X” is the template and all that is required is to copy it.
The preliminary work consists of naming the files with the name of the patient to be treated. For example, in the case of a patient called “Mrs Cunegonde DUPONT”.
Select one of the copies of the file “Mr. X”, rename this copy “cunegonde dupont 01”, open the file: there are three EXCEL files inside; rename them as “repos calcul cunegonde dupont 01” [“cunegonde dupont 01 rest calculation”], “calcul effort cunegonde dupont 01” [“cunegonde dupont 01 exertion calculation”], “resultats cunegonde dupont 01” [“cunegonde dupont 01 results”]
The assignment “repos” [“rest”] or “effort” [“exertion”] must be complied with absolutely as it is presented; above all do not change it . . .
Then open the file “repos calcul cunegonde dupont 01” [“cunegonde dupont 01 rest calculation”], then go to the raw data table (everything will now happen here) and, in the order:
   1. enter the raw data: columns B, C, D and F, G, H
   2. Go to the graph “bolus bruité choix des bornes” [“bolus triggering a signal choice of limits”]
   3. Visually select by eye “temps de debut de calcul” [“start time of calculation” “temps de debut du bolus”[“start time of bolus”] and above all “temps de fin du bolus” [“finish time of bolus”] then the time of “fin du calcul” [“end calculation”];
   4. go to raw data and carefully enter these four times in column 1, also enter the dimensions of the ROIs.
   5. observe the various graphs which show the result,
   6. For the late phase, go to “courbes tardives” [“late graphs”] to select the time; do not forget the dimensions of the ROIs, and the time lapsed between the early injection and the acquisition in the late phase,
   7. Once this is finished, record and open the file called “effort calcul cunegonde dupont 01” [“cunegonde dupont 01 exertion calculation”],
   8. then repeat the same operations
   9. then, all that remains is to open the file “resultats cunegonde dupont 01” [“cunegonde dupont 01 results”]
   10. It is important to note that if, for the same patient, one wishes to carry out another vascular and/or cardiac ROI, everything must be done as if for another patient called “cunegonde dupont 02”

The attached graphs and Table I demonstrate the digital values obtained for a patient by using the method according to the invention in a test which determines the coronary flow reserve (CFR %) after injection of dipyridamole and by using a tracer based on Technetium Tc⁹⁹m

1. A method for the analysis, detection and/or prognosis of disturbances of circulatory flow in the different regions of an organism comprising measuring blood flow before and after a physical, medicinal and/or neurosensory stimulation test, which modifies the blood flow, after administration of a tracer and measuring the fixation and circulation of, the tracer so that its elimination rate is determined with respect to a control value using calculation software.

2. The method for the analysis, detection and/or prognosis of disturbances of the blood flow of claim 1, wherein the target parameter is the coronary blood flow and which consists of measuring said flow before and after administration of a product which modifies the coronary flow or after an exertion, by using a tracer, and measuring the fixation and the circulation of the tracer which allows the determination of a tissue fixation rate with respect to a control value using calculation software.

3. The analysis method of claim 1, wherein the tracer is a gamma ray emitter.

4. The analysis method of claim 1 wherein the gamma ray emitter is a positon emitter.

5. The analysis method of claim 1, wherein the tracer is a fluorescent product.

6. The analysis method of claim 1 wherein the tracer is a contrast product.

7. The analysis method of claim 6, wherein the contrast product is an iodinated product.

8. The analysis method of claim 1 wherein the tracer is a contrast product which is visible by MRI.

9. The analysis method of claim 8 wherein the contrast product which is visible by MRI is a derivative of gadolinium.

10. The analysis method of claim 1 wherein the contrast product is a product which is detectable by ultrasonography or by ultrasonic Doppler effect.

11. The analysis method of claim 1 wherein the fixation of the tracer is measured using an X-ray imaging device, selected from the group consisting of gamma cameras, X-ray scanners, positron emission tomography cameras, and an MRI imaging device.

12. The analysis method of claim 1 wherein the product which modifies the coronary flow is chosen from compounds which increase coronary flow and products which reduce coronary flow.

13. The analysis method of claim 12, wherein the product which increases coronary flow is selected from the group consisting of dipyridamole, adenosine and dobutamine.

14. The analysis method of claim 12, wherein the product which reduces coronary the flow is atropine or its derivative.

15. The analysis method of claim 1 wherein the tracer is a radio-pharmaceutical tracer, a positon emitter or an isotope of thallium.

16. The analysis method of claim 15, wherein the radio-pharmaceutical tracer is selected from the group consisting of derivatives of technetium T_{c}⁹⁹, radioactive isotopes of indium and radioactive isotopes of iodine.

17. The analysis method of claim 15 wherein the radioactive tracer is a tracer labelled with metastable technetium T_{c}⁹⁹, called tetrafosmine.

18. The analysis method of the calculation software is that filed at the Program Protection Agency under the number IDDN FR 001-150011-000-SP2004/00 and called “CORYFLOW”.

19. The analysis method of claim 1 wherein the control value is that of coronary flow reserve, before or after an exertion test or pharmacological treatment.

20. The analysis method of claim 1 wherein the blood flow in the tumorous tissues is measured, before and after injection of a radio-pharmaceutical tracer.

21. (canceled)

22. A method of studying organic disorders linked to vascularization of brain, heart or tumorous tissues in a warm-blooded animal comprising administering to a warm-blooded animal in need thereof a tracer, measuring blood flow before and after a stimulation test and measuring the fixation and circulation of the tracer to determine its elimination rate with respect to a control value with calculation software called CORYFLOW.
